(19) United States
(12) Patent Application Publication
(10) Pub. No.: US 2017/0143540 A1
(43) Pub. Date: May 25, 2017

(54) CONTACT LENS

(71) Applicant: EyeYon Medical Ltd., Nes Ziona (IL)

(72) Inventors: Ofer Daphna, Beit Elazari (IL); Arie Marcovich, Rehovot (IL); Soheil Shaer, Haifa (IL); Nahum Ferera, Petah Tikva (IL)

(73) Assignee: EyeYon Medical Ltd., Nes Ziona, OT (IL)

(21) Appl. No.: 15/422,599

(22) Filed: Feb. 2, 2017

Related U.S. Application Data
(63) Continuation-in-part of application No. 14/814,521, filed on Jul. 31, 2015.
Application No. 14/814,521 is a continuation of application No. 13/785,157, filed on Mar. 5, 2013, now Pat. No. 9,097,915.

(51) Int. Cl. A61F 9/00; G02C 7/04

(52) U.S. Cl. CPC A61F 9/0017; G02C 7/049; A61F 9/00

(57) ABSTRACT

A method for drug delivery includes placing a contact lens over a cornea of an eye. The contact lens includes a central convex disc, a peripheral curved portion that extends radially from the disc, an annular groove formed in the disc, and one or more apertures formed in the groove. An inner volume of the lens defines a chamber. A drug substance is placed in the chamber, which traps the drug substance therein for a period of time.

### CROSS-REFERENCE TO RELATED APPLICATIONS

This application is a continuation-in-part of U.S. patent application Ser. No. 14/814521, filed Jul. 31, 2015, which is a continuation of U.S. patent application Ser. No. 13/785157, filed Mar. 5, 2013, now U.S. Pat. No. 9,097,915, which is incorporated herein by reference.

### FIELD OF THE INVENTION

The present invention relates generally to contact lenses and in particular to contact lenses designed to compensate for an over-hydrated, edematous cornea.

### BACKGROUND OF THE INVENTION

U.S. Pat. No. 8,096,655 to Daphna describes a hyper-osmotic contact lens designed to treat corneal edema. The hyper-osmotic contact lens absorbs fluid from an edematous cornea by the force of an osmotic gradient. The hyper-osmotic contact lens is designed as a microcontainer with walls that are thin relative to its general dimensions, and is shaped as a lens with anterior and posterior walls, which define and bound a hyper-osmotic chamber. The posterior wall, which is the area in which the cornea and the contact lens overlap, is made of a selective water permeable membrane. In this area, water from the edematous cornea can flow out of the cornea into the hyper-osmotic chamber by the force of osmosis, thus dehydrating the cornea itself. The hyper-osmotic chamber may contain a hyper-osmotic transparent medium such as dry hydrogel or solution such as glycerol, salts, etc.

### SUMMARY OF THE INVENTION

The present invention relates to a contact lens, which provides structure different than U.S. Pat. No. 8,096,655, as is described further in detail hereinbelow.

The contact lens can be used to treat corneal edema, wherein the contact lens absorbs fluid from an edematous cornea by the force of an osmotic gradient. The refractive property of the lens can be taken into account according to patient refraction.

The contact lens has other uses, in which osmotic forces are not necessarily needed. For example, the contact lens may be used to trap fluid drugs in the space between the lens and the cornea for retaining the fluid drug for a longer period of time over the eye, thereby increasing the efficacy of the drug.

There is provided in accordance with an embodiment of the present invention a contact lens including a central convex disc, a peripheral curved portion that extends radially from the disc, an annular groove formed at an interior junction of the disc and the peripheral curved portion, and one or more apertures formed in the groove. An inner volume of the lens defines a chamber. A drug substance is placed in the chamber, which traps the drug substance therein for a period of time.

### BRIEF DESCRIPTION OF THE DRAWINGS

The present invention will be understood and appreciated more fully from the following detailed description taken in conjunction with the drawings in which:

FIG. 1 is a simplified front view illustration of a contact lens, constructed and operative in accordance with an embodiment of the present invention.

FIG. 2 is a cross sectional view of the contact lens.

FIG. 3 is an enlarged cross-section of a hole and annular groove of the lens.

FIG. 4 is a simplified side view of the contact lens mounted on an eye, in accordance with an embodiment of the present invention.

FIG. 5 is a simplified side view of a contact lens mounted on an eye, in accordance with another embodiment of the present invention.

### DETAILED DESCRIPTION OF EMBODIMENTS

Reference is now made to FIG. 1, which illustrates a contact lens 10, constructed and operative in accordance with an embodiment of the present invention.

Contact lens 10 includes a central convex disc 12 from which radially extends a peripheral curved portion 14. Central convex disc 12 and peripheral curved portion 14 can have the same curvature or different curvatures. An annular groove 16 is formed at the interior (i.e., posterior side) junction of disc 12 and peripheral curved portion 14. In an alternative embodiment, annular groove 16 may be radially inwards of this junction. In another alternative embodiment, annular groove 16 may be radially outwards of this junction. One or more apertures (through holes) 18 may be formed in groove 16. In an alternative embodiment, the one or more apertures 18 may be radially inwards of annular groove 16. In another alternative embodiment, the one or more apertures 18 may be radially outwards of annular groove 16. In one embodiment, there are 6 apertures 18; in another embodiment there are 8 apertures 18, but the invention is not limited to these numbers.

Central convex disc 12 is made of a material that is oxygen permeable and dimensionally stable for use as a contact lens. A preferred example is G4X p-GMA/HEMA (hioxifilcon D), with water content in the range of approximately 50-75%, commercially available from Benz Research & Development, Sarasota, Fla., U.S. The invention is not limited to this material and other suitable soft or hard materials may be used (e.g., GP PMMA—gas permeable polymethylmethacrylate). For example, the lens may be made of CONTAFLEX GM, commercially available from Contamac Ltd., UK, which is a highly water retentive terpolymer based on glycerol methacrylate.

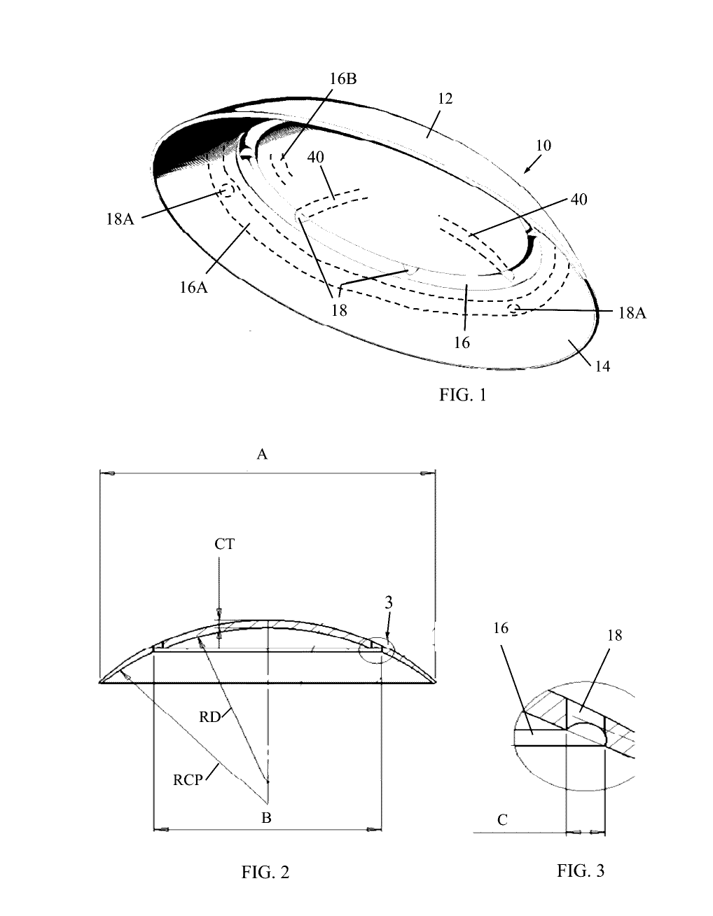

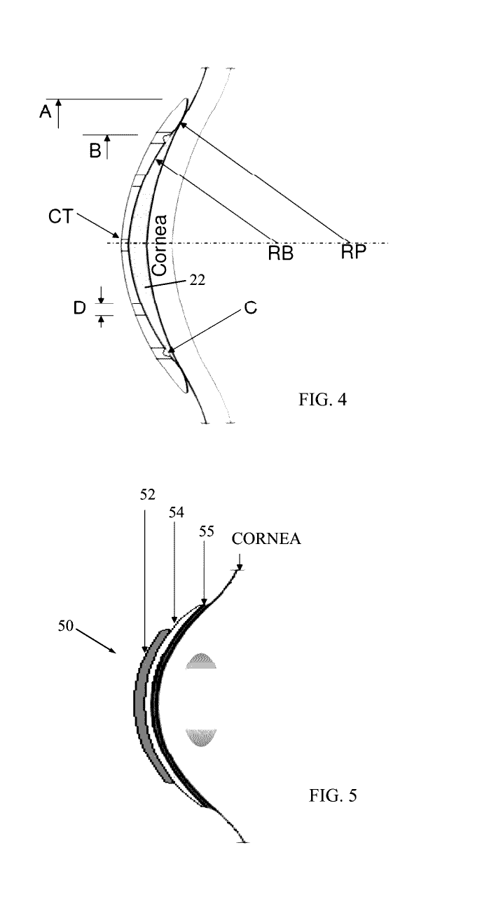

In one embodiment, the inner volume of lens 10, preferably that of central convex disc 12, defines a reservoir 20 (which may be a hyper-osmotic chamber, but in some embodiments is not hyper-osmotic) (seen in FIG. 2) for supporting therein a substance 22 (FIG. 4), which may be a hyper-osmotic transparent medium such as, but not limited to, dry hydrogel, etc., or solution such as, but not limited to, glycerol, salt solution, etc., or non-hyper-osmotic substances, and which may include antibiotics, steroids and other medicinal substances. Substance 22 may also have suitable refraction and transparency properties, which may be selected for modifying vision of a patient.

If substance 22 is hyper-osmotic, substance 22 creates a molecular concentration gradient and thus osmotic pressure gradient between the cornea and hyper-osmotic chamber 20. The osmotic pressure gradient results in a net flow of fluid from the cornea directly into hyper-osmotic chamber 20 by osmosis, thus dehydrating the cornea. Lens 10 can be constructed to reach a steady state net fluid flow or not to reach steady state.

Lens 10 has a suitable volume to enable functioning for a sufficient duration until it is full. Accordingly, contact lens 10 may be used for daily treatment, partial daily treatment or overnight treatment, or any other treatment period which is needed for the patient treatment when it is mounted upon a cornea in an edematous state. Contact lens 10 may be sized to fit over the cornea to the limbus, or alternatively may extend over the limbus.

In another embodiment, chamber 20 is not filled with any substance 22, but instead simply fits over the cornea or cornea and limbus or beyond the limbus. It has been found that the chamber 20 defined by central convex disc 12, even when initially empty (that is, devoid of a hyper-osmotic substance, but, for example, containing air), can create an osmotic pressure gradient that results in a net flow of fluid from the cornea directly into hyper-osmotic chamber 20 by osmosis, thus dehydrating the cornea. A tear film is created, due to the osmotic pressure gradient, between the lens 10 and the cornea. Due to the groove 16 and apertures 18, the tear film creates a surface tension underneath the lens 10 which is relatively trapped and slow to escape. (The apertures 18 are small so the drops do not flow past them but instead are trapped due to surface tension). The entrapped salty tear film increases the hyper-osmotic pressure, which synergistically increases dehydration of the cornea. The structure of the apertures and lens is such that any liquid drop that placed on the exterior surface of the lens will be drawn through the relatively unidirectional apertures 18 to the interior of the lens. Thus, the lens serves as a trap for fluids, such as a hypertonic solution or any other drug.

Optionally, the lens 10 can be heavier at its bottom portion, which may increase the stability of the lens against any torsional movement and maintain the lens in place.

Reference is now made to FIGS. 2 and 4. Non-limiting, exemplary values of lens dimensions are now described.

In one embodiment of the invention, the parameters are as follows:

A overall lens diameter (including peripheral curved portion 14)=11.50 mm

D diameter of aperture 18=0.5 mm

B reservoir diameter (diameter of hyper-osmotic chamber 20)=7.50 mm

C thickness of annular groove 16=0.40 mm

CT center thickness=0.4 mm (see FIG. 3)

RP peripheral eye radius

RB reservoir radius is 0.3 mm steeper than central eye flat meridian

RD radius (curvature) of central convex disc 12=8.60 mm

RCP radius (curvature) of peripheral curved portion 14=8.60 mm

In another embodiment of the invention, the parameters are as follows:

A overall lens diameter (including peripheral curved portion 14)=15.00 mm

D diameter of aperture 18=1.50 mm

B reservoir diameter (diameter of hyper-osmotic chamber 20)=9.50 mm

C thickness of annular groove 16=0.40 mm

CT center thickness=0.25 mm

RP peripheral eye radius

RB reservoir radius is 0.3 mm steeper than central eye flat meridian

RD radius (curvature) of central convex disc 12=7.80 mm

RCP radius (curvature) of peripheral curved portion 14=9.60 mm

In still another embodiment of the invention, the parameters are as follows:

A overall lens diameter (including peripheral curved portion 14)=14.00 mm

D diameter of aperture 18=0.5 mm

B reservoir diameter (diameter of hyper-osmotic chamber 20)=9.50 mm

C thickness of annular groove 16=0.40 mm

CT center thickness=0.4 mm

RP peripheral eye radius

RB reservoir radius is 0.3 mm steeper than central eye flat meridian

RD radius (curvature) of central convex disc 12=8.60 mm

RCP radius (curvature) of peripheral curved portion 14=8.60 mm

In yet another embodiment of the invention, the parameters are as follows:

A overall lens diameter (including peripheral curved portion 14)=11.00 mm

D diameter of aperture 18=0.5 mm

B reservoir diameter (diameter of hyper-osmotic chamber 20)=7.50 mm

C thickness of annular groove 16=0.30 mm

CT center thickness=0.4 mm

RP peripheral eye radius

RB reservoir radius is 0.3 mm steeper than central eye flat meridian

RD radius (curvature) of central convex disc 12=8.60 mm

RCP radius (curvature) of peripheral curved portion 14=8.80 mm

It is noted that in the first and fourth examples, the lens basically covers just the cornea, whereas in the second and third embodiments the lens extends to the limbus and beyond.

As shown in broken lines in FIG. 1, as another option one or more arcuate grooves 40 may be formed at least partially in the spherical longitudinal direction across the inner surface of central convex disc 12. Grooves 40 may extend from apertures 18.

Reference is now made to FIG. 5, which illustrates a contact lens 50, constructed and operative in accordance with another embodiment of the present invention.

Contact lens 50 includes an anterior lens 52 that is mounted over a posterior lens 54, which in turn is mounted on the cornea C of the eye. A tear film 55 may be present between posterior lens 54 and the cornea. Without limitation, anterior lens 52 and posterior lens 54 may be made of G4X p-GMA/HEMA (hioxifilcon D), respectively with 73% and 54% water content; they may be made alternatively of CONTAFLEX GM. Dimensions of anterior lens 52 may be similar to the first above example, whereas posterior lens 54 may be similar to the second above example. The invention is not limited to these values.

The hyper-osmotic contact lens described above is a contact lens that creates a cavity above the center of the cornea. Without limitation, the cavity volume is estimated at ˜10 μl and is generally at a height between 0-200 μm above the corneal surface. This cavity can store substances, such as but not limited to, hypertonic drops, for a long duration and by doing so extracts fluids by osmosis from the cornea.

As mentioned above, the contact lens serves as a trap for fluids, such as a hypertonic solution or any other drug. Accordingly, the contact lens can serve as a drug delivery platform for a variety of drugs.

The contact lens can be used in a variety of applications for drug delivery. The lens provides increased contact time of the medication (any drug substance) with the eye and can reduce symptoms more effectively. For example, the lens may be used to treat recurrent corneal erosion syndrome (RCE), dry eye syndrome, uveitis and certain allergies. The contact lens can also be used in a corneal collagen cross-linking procedure, for intra-operative use and/or reducing procedure time.

What is claimed is:

1. A method for drug delivery comprising:
placing a contact lens over a cornea of an eye, said contact lens comprising a central convex disc, a peripheral curved portion that extends radially from said disc, an annular groove formed in said disc, and one or more apertures formed in said groove, wherein an inner volume of said lens defines a chamber; and
placing a drug substance in said chamber, wherein said chamber traps said drug substance therein for a period of time.

2. The method according to claim 1, wherein said drug substance comprises a hypertonic solution.

3. The method according to claim 1, further comprising extracting fluids by osmosis from the cornea into said chamber.

4. The method according to claim 1, further comprising using said lens to provide increased contact time of said drug substance with the eye.

5. The method according to claim 1, further comprising using said lens to treat recurrent corneal erosion syndrome (RCE).

6. The method according to claim 1, further comprising using said lens to treat dry eye syndrome.

7. The method according to claim 1, further comprising using said lens to treat uveitis.

8. The method according to claim 1, further comprising using said lens to treat an allergy.

9. The method according to claim 1, further comprising using said lens in a corneal collagen cross-linking procedure, for intra-operative use or reducing procedure time.
